(19) United States
(12) Patent Application Publication
(10) Pub. No.: US 2015/0111199 A1
(43) Pub. Date: Apr. 23, 2015

(54) Methods of Using Near Field Optical Forces

(71) Applicant: OPTOFLUIDICS, INC., Philadelphia, PA (US)

(72) Inventors: Robert Hart, Philadelphia, PA (US); Bernardo Cordovez, Philadelphia, PA (US)

(21) Appl. No.: 14/399,600

(22) PCT Filed: Mar. 15, 2013
(86) PCT No.: PCT/US13/32283

§ 371 Date: Nov. 7, 2014

Related U.S. Application Data
(60) Provisional application No. 61/646,574, filed on May 14, 2012.

(51) Int. Cl. G21K 1/00; G01N 21/59; G01N 33/53; G01N 21/47

(52) U.S. Cl. CPC G21K 1/006; G01N 21/47; G01N 21/59; G01N 33/5306; G01N 2201/06113
USPC 435/5; 250/251; 356/72; 436/501; 435/7.1; 435/7.8; 435/6.1; 435/4; 436/547; 436/86; 435/29; 436/94; 436/71; 435/288.7; 422/82.05; 422/82.08

(57) ABSTRACT

Methods of studying, interrogating, analyzing, and detecting particles, substances, and the like with near field light are described. Methods of identifying binding partners, modulators, inhibitors, and the like of particles, substances, and the like with near field light are described. In certain embodiments, the methods comprise immobilizing or trapping the particle, substance, and the like.

### CROSS-REFERENCE TO RELATED APPLICATIONS

The present application claims priority under 35 U.S.C. §119(e) to U.S. Provisional Patent Application No. 61/646,574, filed May 14, 2012, which is hereby incorporated by reference in its entirety.

### BACKGROUND OF THE INVENTION

It is often desired to analyze a substance of interest to determine a property of the substance, including its size, structure, binding activity, and the like. Such analysis is often hampered by the motion of the substances, particularly substances having very small size (e.g. on the nanometer scale). Some techniques require the immobilization of a substance to a solid support. These techniques are intended to keep the substance of interest in a fixed location so that the substance can be sufficiently interrogated and so that the introduction of subsequent compositions (e.g. fluids, etc.) does not alter the localization of the substance.

Immobilization or fixation of the substance often is performed with the use of chemicals or cross-linkers, which can influence the inherent properties of the substance being analyzed. Thus, there is a need in the art for devices and methods to effectively and accurately analyze small substances of interest. The present invention satisfies this need.

### SUMMARY OF THE INVENTION

The present invention provides methods and systems for interrogating at least one property of one or more substances. In one embodiment, the invention provides a method of measuring at least one property of a substance comprising positioning a substance in the vicinity of near-field light of an optical trap, directing light from a light source to the substance, detecting the effect of the light on the substance, and measuring at least one property of the substance based on the detected effect. In certain embodiments, the effect of the light is selected from the group consisting of light scattered by the substance, light emitted by the substance, and light absorbed by the substance. In one embodiment, the method comprises immobilizing the substance at a location using the optical trap, thereby forming a trapped substance.

In certain embodiments, the optical trap comprises at least one structure selected from the group consisting of optical fibers, photonic waveguides, slot waveguides, plasmonic tweezers, photonic crystal resonators, ring resonators, toroidal resonators, Whispering Gallery Mode Resonators, and Fabry Perot resonators.

In certain embodiments, the substance is a substance selected from the group consisting of a molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome.

In certain embodiments, the at least one property of the substance to be measured by the method of the invention is a property selected from the group consisting of size, structure, chemical composition, refractive index, electrical impedance, electrical permittivity, mass, density, temperature, diffusion coefficient, shape, protein folding state, solubility, crystallinity, enzymatic activity, binding activity, binding kinetics, and dissociation kinetics.

In one embodiment, the light source is the near-field light of the optical trap. In one embodiment, the light source is an external light source. In one embodiment, detecting the scattered light comprises detecting the amount of the scattered light. In one embodiment detecting the scattered light comprises detecting the amount and wavelength of the scattered light. In one embodiment, detecting the amount of scattered light comprises the use of a detector selected from the group consisting of a light scattering detector, spectrometer, Raman spectrometer, photodiode, charged coupled device (CCD), spectrum analyzer, interferometer, ellipsometer, integrating sphere, and photomultiplier.

In one embodiment of the method of the present invention, measuring the property of the substance comprises measuring the motion of the substance. In one embodiment, the method comprises releasing the trapped substance.

The present invention also provides a method of measuring the binding activity of a substance comprising, immobilizing the substance at a location using an optical trap, thereby forming a trapped substance, contacting the trapped substance with one or more test substances, and detecting the binding of the strapped substance with one or more test substances.

In one embodiment, the optical trap comprises at least one structure selected from the group consisting of optical fibers, photonic waveguides, slot waveguides, plasmonic tweezers, photonic crystal resonators, ring resonators, toroidal resonators, Whispering Gallery Mode Resonators, and Fabry Perot resonators.

In one embodiment, the trapped substance is a substance selected from the group consisting of a molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome. In one embodiment, the test substance is a substance selected from the group consisting of a molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome.

In one embodiment, the method of the invention measures the binding kinetics between the trapped substance and the test substance. In one embodiment, the method measures the binding affinity between the trapped substance and the test substance.

In one embodiment, at least one of the trapped substance and test substance are labeled with a detectable label, and the method comprises detecting a detectable signal from the detectable label. In one embodiment, the detectable label is selected from the group consisting of fluorescent labels, radioactive labels, ferromagnetic labels, paramagnetic labels, luminescent labels, electrochemiluminescent labels, phosphorescent labels, mass labels, Raman labels, molecular beacons, upconverting phosphors and chromatic labels.

In certain embodiments, the test substance is contacted with the trapped substance by flowing the test substance to the trapped substance.

The present invention also provides a method of identifying a modulator of a substance comprising immobilizing the substance at a location using an optical trap, thereby forming a trapped substance, contacting the trapped substance with one or more test substances and measuring a property of the trapped substance, wherein a change in the property of the trapped substance when contacted with the test substance indicates that the test substance is a modulator of the substance.

In one embodiment, the optical trap comprises at least one structure selected from the group consisting of optical fibers, photonic waveguides, slot waveguides, plasmonic tweezers, photonic crystal resonators, ring resonators, toroidal resonators, Whispering Gallery Mode Resonators, and Fabry Perot resonators.

In one embodiment, the trapped substance is a substance selected from the group consisting of a molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome.

In one embodiment, the test substance is a substance selected from the group consisting of molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome.

In one embodiment, the property of the trapped substance is a property selected from the group consisting of size, structure, chemical composition, enzymatic activity, binding activity, binding kinetics, and dissociation kinetics.

In one embodiment, the test substance is contacted with the trapped substance by flowing the test substance to the trapped substance.

The present invention also provides a system for measuring a property of a substance comprising at least one optical trap; and at least one detector for measuring the property of the substance. In one embodiment, the system comprises a microfluidic delivery system. In certain embodiments, the system comprises an external light source.

In one embodiment, the system comprises at least one optical trap comprises at least one structure selected from the group consisting of optical fibers, photonic waveguides, slot waveguides, plasmonic tweezers, photonic crystal resonators, ring resonators, and toroidal resonators. 33. In one embodiment, the at least one optical trap comprises at least one power source. In one embodiment, the power source is an optical power source configured to provide optical power to the optical trap.

In one embodiment, the system comprises at least one detector comprises a detector selected from the group consisting of fluorescence microscopes, fluorescence detectors, fluorescence spectrometers, light scattering detectors, optical sensors, Raman microscopes, Raman spectrometers, spectrometers, photodiodes, charged coupled devices (CCDs), Complementary metal-oxide-semiconductor (CMOS) cameras, spectrum analyzers, interferometers, ellipsometers, integrating spheres, and photomultipliers.

In one embodiment, the system comprises at least one sensor selected from the group consisting of quartz crystal microbalances, cantilevers, electrochemical sensors, acoustic sensors, thermal sensors, impedance sensors, and whispering gallery mode optical sensors.

In one embodiment, the at least one optical trap is patterned on substrate selected from the group consisting of a silicon substrate, glass substrate and polymer substrate.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic depicting an exemplary method of detecting a property of a trapped particle using the near-field light.

FIG. 2 is a schematic depicting an exemplary method of detecting a property of a trapped particle using externally sourced light.

FIG. 3 is a schematic depicting the relative motion of differently sized trapped particles.

FIG. 4 is a schematic depicting the use of an optical trap to detect binding between two substances.

FIG. 5 is a schematic depicting the use of optical traps in exemplary immunoassays.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, an “optical trap” refers to an attractive force produced by light energy, either in free space or caused by a nanostructure, which is used to physically hold and manipulate nanoscale and microscale objects.

As used herein, “near field light” refers to the passage of light in sub wavelength dimensions. In some instances the near field effect discussed herein is known as the “evanescence wave”.

As used herein “evanescent wave” or “evanescence” refers to a type of non-propagating light form that rapidly decays from a higher refractive index material to a lower one.

As used herein, a “photonic waveguide” refers to a light guide patterned in microfabricated material that has microscale or subwavelength dimensions.

As used herein, a “slot waveguide” refers to an optical waveguide that guides strongly confined light in a sub-wavelength-scale low refractive index region by total internal reflection. In certain embodiments, a slot-waveguide comprises two strips or slabs of high-refractive-index separated by a sub-wavelength-scale low-refractive-index slot region of a lower refractive index material. In some embodiments, the lower index material is an aqueous solution or buffer.

As used herein, “plasmonic tweezer” refers to a nanoscale optical trapping system that exploits plasmonic resonance to enhance the electric field and enable stronger trapping forces.

As used herein, “photonic crystal” refers to a periodic optical nanoscale structure formed by 2 or more materials of varying refractive indexes.

As used herein, “photonic crystal resonator” refers to a photonic crystal that exhibits resonance when light constructively interferes in its structures. If light is continuously sourced to the photonic crystal, the crystal or a portion of the crystal will exhibit larger optical intensity than the incoming light.

As used herein a “ring resonator” refers to a waveguide in a closed loop coupled to one or more input/output waveguides. When light is supplied to the ring resonator at the correct wavelength(s) the light is positively interfered and the optical intensity in the ring is enhanced. A “toroidal resonator” refers to a ring resonator as defined above but where the walls of the resonator are rounded with a cross-sectional geometry closer to that of a circle as opposed to a rectangle.

As used herein “Whispering Gallery Mode Resonator” refers to concave structures where light traveling in the periphery of the structure forms a constructive interference pattern that leads to resonance.

As used herein “Fabry Perot resonator” refers to an optical resonator formed by two parallel reflecting mirrors separated by medium (can be a gain medium) in between. A standing wave is formed between the reflecting mirrors, leading to a resonance condition and thus light amplification inside the cavity.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides methods of analyzing a property of a substance using near field light. For example, a biological, chemical, and/or physical property of a substance of interest can be measured using the embodiments of the present invention. For example, in certain embodiments, the method comprises using near field light to interrogate a substance. In certain embodiments, the method of the invention comprises directing light from a light source to the substance, where the property of the substance is directly or indirectly measured based on a detected effect of the light on the substance. For example, the property of the substance, in certain embodiments, comprises at least one of detecting the light scattered by the substance, detecting the light emitted by the substance, or detecting the light absorbed by the substance.

In one embodiment, the light source is the near-field light from an optical trap. In another embodiment, the light source is an external light source. In some embodiments, the method comprises using near field light, for example, from an optical trap, to immobilize or trap the substance. However, in some embodiments, the substance is not trapped. Rather, the substance is positioned in the vicinity of near field light. For example, the substance can be positioned within or nearby near-field light. As used herein, positioning a substance refers to placing the substance in a stationary position and to applying a moving particle, for example using fluidics. Thus, while the present invention is exemplified herein using trapped substances, those skilled in the art would appreciate that the methods described herein are equally applicable to substances that are not trapped.

The methods can be used, for example, to study, the size of substance, the structure of a substance, the chemical composition of the substance, the kinetics of an enzyme, binding affinities between two or more substances of interest, testing whether a compound is an agonist, antagonist, or other type of modulating compound (e.g. activator, potentiator, or inhibitor). The method can also be used to measure the refractive index, electrical impedance, electrical permittivity, mass, density, temperature, diffusion coefficient, shape, protein folding state, solubility, or crystallinity of a substance.

In some embodiments, the present invention provides methods of immobilizing agents of interest so that they can be studied or reacted with other reagents. In some embodiments, the immobilization is performed without the use of chemicals or cross-linkers. The immobilization can be done, for example, using optical forces combined with fluid dynamics, i.e. optofluidics.

For example, rather than using chemical means, in some embodiments, the method comprises the use of optical forces to trap, capture, affix, or immobilize substances (e.g. nucleic acid molecules, proteins, enzymes, antibodies, viruses, bacteria, cells, small molecules, particles, bioparticles, nanotubes, quantum dots, protein complexes, carbohydrates, lipoparticles, vesicles, microparticles, oil droplets, and the like) or other very small particles and keep them in the same location. By localizing them to a known location, the substance can easily be interrogated and the substance's surroundings can easily be modified. For example, the solution and/or the reagents that the substance comes into contact with can be modified without altering the substance itself.

Previously, it has been shown that the location and movement of particles can be manipulated using optical forces. For example, U.S. Patent Application Publication Nos. 2011/0039730 and 2012/033915 and International Application Publication No. WO/2012/048220 describe various devices and methods that can be used to manipulate the location and movement of particles, each of which is hereby incorporated by reference in its entirety. By generating optical forces of sufficient intensity, it is possible to trap proteins and other nanoparticles into one physical location. Accordingly, optical trapping can take the place of chemical immobilization or the use of other molecules/substances to affix a particular substance at a physical location.

Once substances are captured within the optical trap, new reagents can be introduced and made to interact with the captured substances. At any given time, the trap can be turned off and the substances will be free to diffuse away or be driven away by flow. Therefore, optical trapping can be used to study the properties of substances alone or in the presence of other reagents. The embodiments described herein may make reference to substances that are trapped in or on an optical trap. In addition to substances, such as molecules, compounds, enzymes, proteins, peptides, organic molecules, inorganic molecules, nucleic acid molecules, and the like, particles or other substances, such as nano- or micro-particles (e.g. quantum dots, nanotubes, microspheres) can also be trapped by an optical trap. These particles may be made up of plastic (e.g. polystyrene) and then coated with another substance. These other types of particles, therefore, can also be used and substituted where the term “substance” is used. Additionally, viruses, virus-like particles, bacteria, vectors, cells, liposomes, which may be referred to as bioparticles, and the like can also be trapped and manipulated according to the methods described herein. Other types of substances that can be trapped include liposomes or liposomal structures. Thus, the term “optically trapped substance” refers to any molecule, compound, composition, particle, or biological composition that can be optically trapped using an optical trap as described herein and elsewhere.

The embodiments provided herein have significant and unexpected advantages over previously used methods to study substances. For example, previously a substance could be affixed to a plate by either cross-linking or by binding it directly to another substance (e.g. an antibody). These traditional methods of affixing, however, will affect the properties of the native substance being studied. For example, it will restrict the movement and surface area of the substance being studied such that it will no longer behave as it does in solution. For example, the substance's movement will be dramatically constrained, parts of the substance can be hidden due to steric hindrance, the substance can denature, the substance can be partially blocked or mis-oriented, and the substance can experience a variety of electrical forces that arise from its proximity to the surface.

Accordingly, the previously used methods that rely upon methods of trapping other than optical trapping can result in altered properties, decreased functionality, and/or decreased performance. The embodiments described herein overcome these disadvantages and provide superior methods because optical trapping should not result in altered properties, decreased functionality and/or decreased performance of the substance being studied. Optically trapped substances will have better performance because they will behave in a more physiological nature, since the substances will still be freely moving about in solution.

Accordingly, in some embodiments, the following methods are provided as described herein and in the claims. The presently described methods have advantages over previously described methods because they should require little or no optimization and little or no prior knowledge of the substance or protein to be immobilized in order to specifically tailor the immobilization. For example, it is often desirable for a surface conjugation to be targeted to a specific site on a protein in order to properly orient a specific region of the protein away from the surface. If the surface of interest is not exposed and is hidden by the chemical attachment then an assay studying the surface cannot be performed. This must be determined on a case by case basis when using chemical methods to affix a substance to a particular location. In contrast, optical trapping of a substance does not need to be done on a case by case basis to determine if the assay can be performed because the optical trapping will allow enough of the surface of interest to be exposed. The present methods can be used with any substance provided that the substance can be captured using optical trapping.

The present methods also have other significant advantages over previous methods. For example, in previous methods immobilization of a substance requires numerous, labor, and time intensive steps. For example, these steps may include activating the surface or the substance. In contrast, using optical trapping to immobilize a substance to a particular location can be done in a single step. The surface does not need to be activated by a chemical or other means. In some embodiments, optical trapping does not require a chemical step to activate the surface. In some embodiments, optical trapping a substance to a surface does not require coating the surface with a binding partner (e.g. antibody) to trap the substance. For an optical trap, the substance is immobilized to a surface by turning on a laser or optical power source. Once the laser or optical power source is turned on the substance is affixed to the location. In certain embodiments, the laser is tuned to particular wavelength to immobilize a substance.

As used herein, the terms “affixed” or “immobilized” refers to a substance that can be held at a particular position. The substance can be affixed temporarily or permanently. Additionally, the substance can be affixed to a particular position on the optical trap in such a manner that allows the substance to change orientation. Affixing the substance, refers to the substance being held at a particular position, but that the substance may still be able to move within the constraints of the optical trap. For example, a substance that is being optically trapped with a trap duty cycle is still considered to be affixed to the optical trap.

Another advantage of the presently described methods over previous methods is that the optically trapped substance(s) can be easily eluted (i.e. released) from the trap without the use of harsh chemicals or other reagents. For example, in some embodiments, an optically trapped substance is released by turning off the laser or optical power source that is trapping the substance. In certain embodiments, an optically trapped substance is released by tuning the laser to a different wavelength. Once the optical force is diminished the substance can be eluted and/or purified for further analysis. In contrast, in previous methods, substances that are chemically attached to a surface can only be released with the use of harsh chemicals, harsh salt conditions, enzymatic cleavage, or the use of other reagents. These reagents need not be used to elute or release an optically trapped substance. Accordingly, the presently described methods provide a superior method of releasing, eluting, or purifying a trapped substance because of these advantages.

In some embodiments, the orientation of the trapped substance is not fixed. For example, by modulating the optical force through the use of a trap duty cycle, the substance can be allowed to change orientation while still being affixed to a particular location. This is contrast to previous methods, where once a substance is trapped to a location through either chemical attachment or by binding to another binding partner, the orientation of the substance is more or less fixed and does not change. In contrast, optical trapping and the modulation of the optical forces can keep the substance affixed to a particular location but also allow for the orientation of the substance to change. This advantage better mimics how a substance would behave under physiological conditions or how the substance would behave in a cellular environment.

In certain embodiments, the optical trapping can be used with different optical traps of varying strengths. Therefore, where a complex or mixture of substances is optically trapped on a surface by modulating the optical force, different substances can be selectively captured and/or eluted. This is unlike previously described methods.

The presently described methods can be used in conjunction with other detection methods. The detection methods can be used to measure substance size, molecule composition, binding affinities, kinetics, inhibition or activation of an enzyme or other process, and the like. Examples of other detection methods include, but are not limited to, fluorescence, chemiluminescence, optical scattering, Raman spectroscopy, colorimetric, electrochemical methods and Surface Plasmon Resonance and spectroscopy. Such exemplary methods may be integrated onto devices or systems comprising an optical trap (i.e. on-chip), or alternatively be externally coupled (i.e. off-chip).

The materials used to perform certain embodiments of the methods and assays described herein can be any optical trap. Examples of optical traps are described in U.S. Patent Application Publication Nos. US2011/0039730 and US2012/033915 and International Application Publication No. WO/2012/048220, each of which is hereby incorporated by reference in its entirety. Exemplary optical traps may comprise photonic structures and/or optically resonant structures. For example, photonic structures include, but are not limited to, optical fibers, photonic waveguides, slot waveguides, plasmonic tweezers, and combinations thereof. Optically resonant structures include, but are not limited to, photonic crystal resonators, ring resonators, toroidal resonators, and combinations thereof. In one embodiment, the optical trap comprises a near field optical structure, which produces an evanescent field to optically trap a substance or particle of interest. Optical traps used herein are capable of trapping substances of any size or shape, including substances that would otherwise be too small to trap.

In one embodiment, an optical trap used in the method of the invention is capable of trapping or immobilizing a substance having size less than 1 μm, in any one dimension. In one embodiment, an optical trap used in the method of the invention is capable of trapping or immobilizing a substance having size less than 500 nm, in any one dimension. In one embodiment, an optical trap used in the method of the invention is capable of trapping or immobilizing a substance having size less than 100 nm, in any one dimension. In one embodiment, an optical trap used in the method of the invention is capable of trapping or immobilizing a substance having size less than 10 nm, in any one dimension.

In one embodiment, the optical trap comprises one or more slot waveguides (US2012/0033915). In brief, a slot waveguide comprises a nanoscale slot having a relatively low refractive index, sandwiched between two walls of significantly higher refractive index. A laser provides light within the slot, which produces an optical trapping force to immobilize a molecule or particle within or on the sides of the slot. In another embodiment, the optical trap comprises one or more photonic crystal resonators, positioned near the one or more photonic waveguides. Light within the waveguide, tuned to the resonant wavelength, is evanescently coupled into the resonator and produces a concentrated optical trapping force at or near the photonic crystal resonator (WO2012/048220). In one embodiment, the resonator is placed in-line with the waveguide. In this case, rather than having the resonator positioned in close proximity to the waveguide such that the light evanescently couples in, the waveguide itself has the resonator built directly into its material. While particular embodiments are exemplified herein using waveguides and/or photonic crystal resonators, one skilled in the art would recognize that any suitable optical trap can be utilized in the method of the present invention.

The optical trap can be powered by a powering system. The powering system can be a laser or other type of optical force. In one embodiment, the power of the laser is configured to be between 1-1000 mW. In another embodiment, the power of the laser is configured to be between 10-100 mW. In certain embodiments, the power of the laser and/or the wavelength of the light determines the size or the size range of the trapped substance. For example, in one embodiment, the power of the laser is tuned such that only substances of a particular range are trapped by the optical trap, while substances that are larger than the range or smaller than the range flow past the trap.

The optical trap can also be combined with a fluidic delivery system such that one or more molecules are particles are delivered to the optical trap. This can be done with a fluidic channel or flow cell. For example, in one embodiment, the optical trap and fluidic delivery system are included on a single chip. In another embodiment, the optical trap is applied to a chip, comprising a fluidic delivery system.

In some embodiments, a substance of interest (i.e. molecule or particle) is contacted with an optical trap to immobilize the substance. The optical trap can be turned on prior to or after the substance is contacted with the optical trap. In one embodiment, the substance is contacted with the optical trap by allowing the substance to flow over the optical trap. Any method can be used to allow the substance to flow over the optical trap. For example, any fluidics system can be used. For example, a syringe can be used to apply the substance over the optical trap. However, much more sophisticated systems can be used such that the system can be automated.

In some embodiments, the flow rate of the fluid carrying the substance can be modulated (e.g. increased or decreased), which can facilitate the trapping or elution of certain substances from the optical trap. Additionally, different solutions can be used in different traps by using separated channels. The flow can be used to affect how the substance is optically trapped. In another embodiment, the flow can be used to influence which specific type of substance, from a fluid comprising a plurality of substances, is optically trapped. For example, in one embodiment, the flow is tuned such that only substances of a particular range are trapped by the optical trap, while substances that are larger than the range or smaller than the range flow past the trap.

A number of different flow schemes can be used, including but not limited to, pressure driven flow, electromagnetically driven flow, electrokinetically driven flow, capillarily driven flow, flow focusing, flow contacting, varying channel geometries to affect how the particle or substance of interest is optically trapped.

In some embodiments, the fluid delivery scheme can comprise of one or more inlets and one or more outlets. Additionally, a microfluidic circuit can be established to precisely meter and deliver different fluids to the trapping region. Each inlet and channel can deliver separate reagents or process reagents, for example to microfluidically generate a concentration gradient.

As described herein, the trap can be powered before introducing the sample that contains the substance of interest or the trap can be turned on after the sample has been introduced. The trap can also be pulsed or modulated such that there is a controllable duty cycle. That is, the power can be turned on and off rapidly according to some periodicity. Thus, the trap will alternate between an on state and an off state with some frequency. The power of the trap can also be modulated by simply reducing the optical power delivered to the trap, by adjusting the polarization of the light or by changing the wavelength of the light. The power of the trap can also be adjusted in order to trap smaller objects. The amount of force that is necessary can be determined by the skilled artisan in view of the present disclosure. In some embodiments, the power can be pulsed to prevent substances from sticking to the surface (e.g. to avoid surface charges and other surface effects).

Examples of substances that can be trapped include, but are not limited to, proteins, particles (e.g. polystyrene or other plastics) bioparticles, enzymes, nucleotide sequences (DNA, RNA, mRNA, and the like), organic molecules, inorganic molecules and the like. The substance can be any substance of interest that is capable of being trapped by an optical trap.

In some embodiments, the method uses a measurement system or measurement detection device. For example, the optical trap may be combined with known devices and techniques to interrogate the trapped molecule to determine its size, composition, activity, binding affinities, kinetics, inhibition or activation of an enzyme or other process, and the like. Exemplary devices include, but are not limited to, fluorescence microscopes, fluorescence detectors, fluorescence spectrometers, light scattering detectors, optical sensors, Raman microscopes, Raman spectrometers, spectrometers, photodiodes, charged coupled devices (CCDs), Complementary metal-oxide-semiconductor (CMOS) cameras, spectrum analyzers, interferometers, ellipsometers, integrating spheres, and photomultipliers.

In some embodiments, the optical trap is treated with an agent to block or functionalize the surface. In some embodiments, the optical trap is not treated with an agent to functionalize the surface. In some embodiments, the optical trap is not treated with an agent to block (e.g. BSA, gelatin, and the like) the surface.

In some embodiments, multiple optical traps can be used on the same substrate concurrently or at different times.

In some embodiments, after, prior, or simultaneously to the substance being optically trapped, additional reagents can be introduced to the system to interact with the system walls or trapped substance themselves. These reagents can be any reagent that can be used in the optical trap system. Non-limiting examples, including, blocking buffers to passivate the surface so that future reagents do not non-specifically bind a small molecule that may or may not bind to the trapped substance, additional particles that may or may not bind to the trapped substance, a washing buffer to remove the uncaptured particles, a new buffer or solution, a continuously changing buffer (varying in concentration of salts, pH, concentration of buffer constituent), and the like.

In one embodiment, the method comprises application of an additional reagent comprising one or more additional particles (e.g. bioparticles, proteins, organic molecules, inorganic molecules, nucleotide molecules, and the like) to evaluate their influence on the trapped substance. In one embodiment, the additional reagent comprises one or more additional particles that may or may not influence the size or activity of the trapped substance. In one embodiment, these particles can bind to the trapped substance and form temporary or permanent complexes or aggregates. In some embodiments, the additional particle(s) can cause the trapped substance to aggregate/form complexes or cause the trapped substance to become disassociated into smaller components or subunits.

For example, in one embodiment, the method of the invention comprises a screening method, where additional reagent comprise a test compound from a library of compounds, and the optical trap is used to evaluate whether or not the test compound influences the properties of a trapped substance.

In one embodiment, the added particle can alter the activity of the trapped substance. For example, the trapped substance's activity can be activated, deactivated, inhibited, up-regulated, down-regulated in the presence of one or more additional particle. The additional particle, for example, can be a test compound from a library of compounds and the trapped substance can be an enzyme. Therefore, the enzyme's properties can be measured in the presence or absence of the test compound to determine if it affects the properties and function of the enzyme.

The substance activity can be measured by detecting a signal. The signal can be visual, colorimetric, fluorescent, radioactive, or the like. Any detectable signal can be used.

In some embodiments, the trapped substance is eluted. The trapped substance can be eluted by turning off the optical trap, modulating the power of the optical trap, or tuning the laser of the optical trap to a different wavelength. The elution may also elute other compounds that are bound to the trapped substance. The eluted trapped particle or complex can then be analyzed or otherwise manipulated.

In some embodiments, the environment of the trapped substance is altered. In some embodiments, the temperature of the trapped substance is modified. In some embodiments, the trapped substance is exposed to light (any wavelength), radiation, electric field, gasses, or other reagents to change the substances environment. The trapped substance can also be trapped for a specific period of time by modulating the field that is trapping the substance.

The presently described methods can be used for many methods and uses. For example, the presence of trapped substances, the rate of binding/unbinding of reagents/chemicals/proteins/molecules etc. in solution to trapped substances, the reaction rate of trapped substances; the disassociation of trapped complexes; the size of the trapped substances; the composition of trapped substances; the diffusion coefficient measurement of the substances, can all be measured. The measurements can be done by, for example, photoluminescence (e.g. Fluorescence); chemiluminescence; colorimetric; spectroscopic; using an external system; using a system built onto a chip; electrochemical methods; or any physical, chemical, biological, optical, or electrical methods.

In some embodiments, a control is used. Control measurements can also be taken of other traps that have different particles or other control measures. Measurements can be taken of other locations on the chip for background or control purposes. In some embodiments, no measurement may be made in conjunction with the trapped particle while the particle is trapped.

### Trapped Substance Interrogation

In one embodiment, the method of the invention comprises interrogating a substance to determine a property of the substance. In one embodiment, the substance is trapped. Exemplary properties of the substance, attainable by the present method include, but is not limited to, size, quantity, composition, concentration, refractive index, and density. As described elsewhere herein, the substance may be any suitable substance that may be interrogated using near field light. Exemplary substances include, but are not limited to, enzymes, proteins, small molecules organic molecules, inorganic molecules, nucleic acid molecules, nanoparticles, microparticles, microspheres, quantum dots, and the like. In one embodiment, the method is used to determine the presence and/or properties of a particular substance in mixture. For example, in one embodiment, the method is used to evaluate the presence and/or properties of contaminants that would otherwise be nearly impossible to determine.

In certain embodiments, the interrogation method of the invention utilizes one or more optical traps to trap or immobilize a substance. For example, in one embodiment, a solution comprising a substance of interest is applied in the vicinity of the trap. Once a substance is trapped, one or more detection mechanisms are used to obtain information about the trapped substance. The optical trap produces a trapping force, immobilizing the substance in a precise and known location. Measurement techniques can therefore be focused on, aimed at, or constructed near the optical trap which will give highly specific and sensitive measurements. Trapping the substance in place allows increased measurement (integration) times. Measuring the substance for a longer amount of time increases the sensitivity of the selected detection mechanism. This method allows for the highly specific and sensitive measurement of a single substance.

As described elsewhere herein, the optical trap may comprise near-field optical structures, including, but not limited to, photonic wave guides, photonic crystal resonators, slot waveguides, and photonic tweezers. In one embodiment, the method comprises using the light from the near field optical structure itself to interrogate the particle. In another embodiment, the method comprises application of light from an external light source (i.e. separate from the near field light) to a trapped substance to interrogate the substance. In another embodiment, the dynamic motion of the trapped substance under the influence of the near field optical trap is correlated to fundamental substance properties. The motion can be measured through a variety of means to elucidate these properties.

Interrogation of Trapped Substance Using Interrogation Light from Optical Trap

In one embodiment, the near field light can be used to interrogate the substance of interest. In certain embodiments, the near field light is from an optical trap. In certain embodiments, the near field light is used to interrogate a trapped substance. In some embodiments, the trapping force is enhanced if an optical resonator is used. In certain embodiments, using the light from the trap is advantageous, because this light is highly confined and only substances within the evanescent wave are subjected to this interrogation. Since the evanescent field is so tightly confined, nearby particles do not generally interfere with the measurement, which can therefore be highly specific to the trapped substance. This allows the method to be highly specific. Further, there are also advantages associated to signal to noise. Because the interrogation light is mainly contained inside the waveguide, there is less background light around the trapped substance, thereby improving the ratio of signal to background.

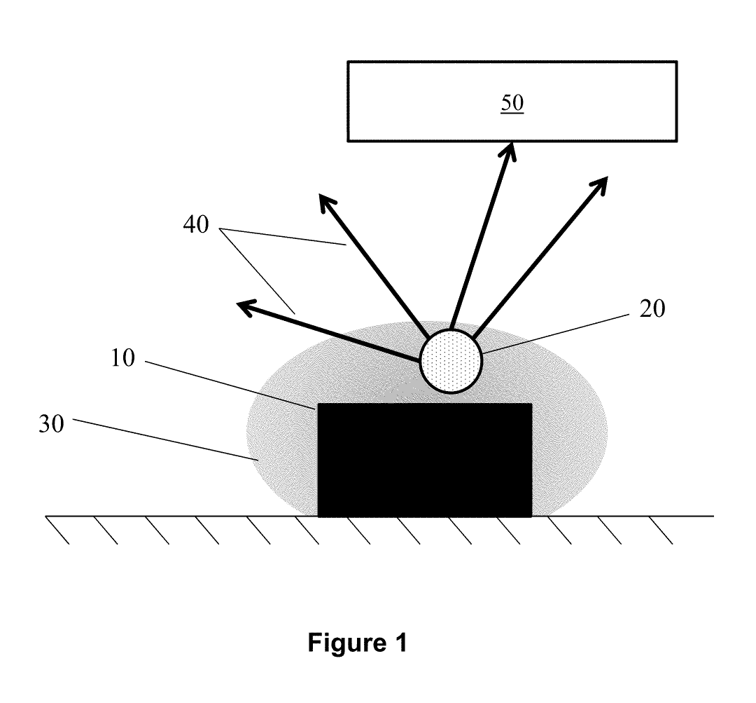

FIG. 1 depicts an exemplary schematic of a method of using interrogation light from the optical trap to interrogate a trapped substance. As depicted, optical trap 10 is used to immobilize a trapped substance 20. In one embodiment, optical trap 10 is a waveguide. As described elsewhere herein, exemplary optical traps used in the present invention utilize a laser positioned to provide light within a narrow slot of a slot waveguide to produce an evanescent field to trap substance 20. In the method depicted in FIG. 1, the near field light 30, which is the light emanating from optical trap 10, is used to interrogate substance 20. In one embodiment, a property of substance 20 is determined by measuring the amount of scattered light 40 caused by substance 20. The presence of substance 20 within the evanescent field of optical trap 10 causes some of the light to be converted, through scattering, into far-field light, which propagates and reaches one or more detectors 50. Larger substances cause more light to be scattered while small particles cause less. In one embodiment, detector 50 is positioned on a chip comprising optical trap 10 and, optionally, a fluidic delivery system. In another embodiment, detector 50 is off-chip (i.e. not located on the same chip as optical trap 10).

Following Rayleigh scattering (substance much smaller than incident wavelength), the substance scattering cross section (σₛ) is given by:

$${σ}_{s} = \frac{2 {π}^{5}}{3} \frac{{d}^{6}}{{λ}^{4}} {( \frac{{n}^{2} - 1}{{n}^{2} + 2} )}^{2} ,$$

where λ is wavelength, n is the refractive index of the particle, and d is the diameter of the substance. Thus, the scatter cross-section scales with substance diameter to sixth power. For example, assuming a 0.2 μm substance and a 0.1 μm substance come to the vicinity of the optical trap (for example, 2 viruses of different sizes), the larger substance will scatter ˜64 times more than the smaller substance. Thus, measuring the scattering cross-section is very selective to size. Further, as scattering cross-section also scales to (1/λ)⁴, the ability to discriminate substances by scattering cross-section may, in certain embodiments, be enhanced by optimizing the wavelength. For example, if a green laser (532 nm) is used to probe instead of an NIR laser (1064 nm), approximately 16 times more scattering on the substance is obtained.

In certain embodiments, the scattered light is used to get a spectrometry or Raman spectrometry signal from the trapped substance. For example, the scattered light is captured and detected by a spectrometer. In certain embodiments, the spectrometry signal is used to determine what type of substance the trapped substance is. For example, in certain embodiments, the signal is used to determine if the substance is a protein, small molecule, metal contaminant, or the like. In some embodiments, the signal is used to determine if the substance is a protein aggregate.

In one embodiment, a property of the substance is determined by measuring the output light from a waveguide. The light that is guided along the waveguide is altered due to the presence of a substance. Larger particles scatter more light by having a larger interaction length with the waveguide, resulting in less light coming out of the waveguide. In certain embodiments, where an optical resonator is used, the presence of the substance near the resonator causes the resonance wavelength to shift, since its presence changes the local refractive index dramatically. Both a power reduction due to scattered light and a shift in resonance wavelength can be measured at the outlet. Resonance shift can be measured by sweeping the input laser wavelength and collecting the light after it passes through the resonator and then building a power spectrum. In one embodiment, a power change is observed at the output using a single wavelength. In another embodiment, many wavelengths are inputted simultaneously

In certain embodiments, it is advantageous to use the near field light from the optical trap to interrogate the substance because the energy can be made more intense compared to far field methods, using an external light source, without incurring high system costs, encountering safety concerns or damaging samples. A safer, lower powered laser can be used because this method of waveguide-based interrogation greatly confines and amplifies the optical energy. In contrast, exposing a larger area, with an external source, to a similar amount of energy density would require a more powerful laser, which will be more expensive and likely more dangerous to the probed substances and user. As a result, in certain embodiments, this interaction is more efficient than with far field methods. Further, because of scaling laws, smaller spots of light dissipate heat more easily than larger spots of light. Interrogating the sample with far field light from an external source and attaining the same optical energy density would cause more heating which could damage the sample, distort measurements, or harm the device itself.

Interrogation of Trapped Substance Using Interrogation Light from External Source

In one embodiment, the method comprises applying an interrogating light from an external light source (i.e. off-chip) to a substance. In certain embodiments, the substance is a trapped substance, as described herein. Small particles suspended in fluids move around randomly due to Brownian motion. However, in certain embodiments, using the methods and devices described herein, a substance subjected to a near field optical trap is localized to a known location for an extended period of time. This allows measurement techniques to be focused on, aimed at, or constructed near the particle trap for time periods not otherwise attainable in dynamic systems. This allows for highly specific measurements that can be time averaged or integrated for greater sensitivity.

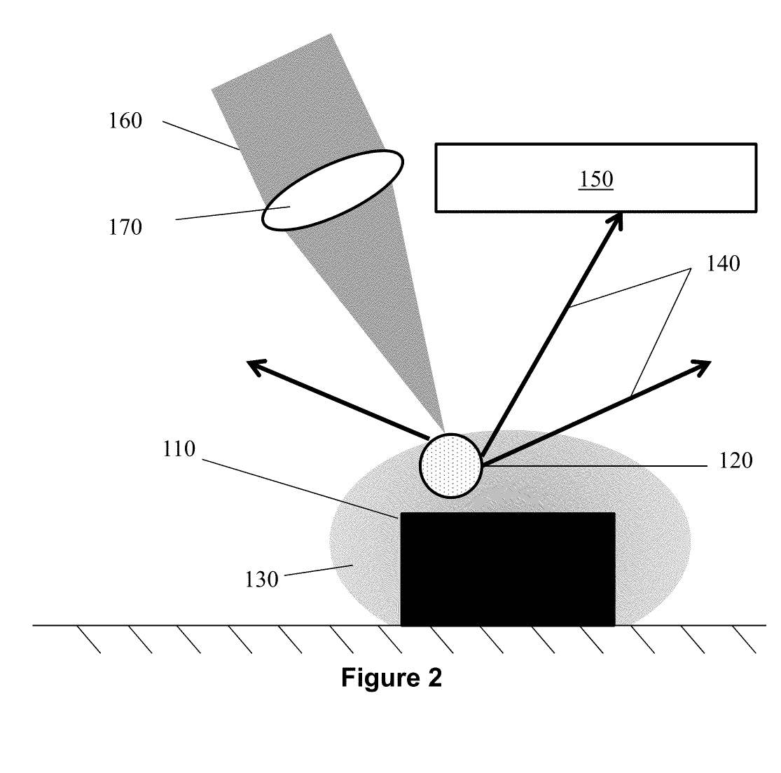

FIG. 2 depicts an exemplary schematic of a method of using interrogating light from an external source to interrogate a trapped substance. As depicted, optical trap 110 is used to immobilize a trapped substance 120. In this method, interrogating light 160 from an external source is used to determine a property of substance 120, instead of using near field light 130 from optical trap 110. In certain embodiments, interrogating light 160 is focused upon substance 120 by use of a focusing lens 170. For example, in one embodiment, the method uses a conventionally focused laser (as opposed to one coupled to a waveguide) to interrogate the trapping region. Scattered light 140 is collected and measured by detector 150. In some embodiments, detector 150 is a spectrometer. Trapped substance 120 stays within the trapping region allowing signals to be collected until the substance is actively released. Non-trapped substances may occasionally enter and exit the interrogation region but will have a much smaller time-averaged impact on the overall measurement.

This method can also be exploited using locally micro or nano-structured sensing methods. For example, in one embodiment, the method comprises detecting a property of the trapped particle using an electrochemical sensor, which can consist of several electrodes patterned near the optical trap. Other exemplary sensing methods include, but are not limited to, optical, acoustic, mechanical, thermal, and the like.

### Interrogation of Trapped Substance Using Position and Motion of Substance

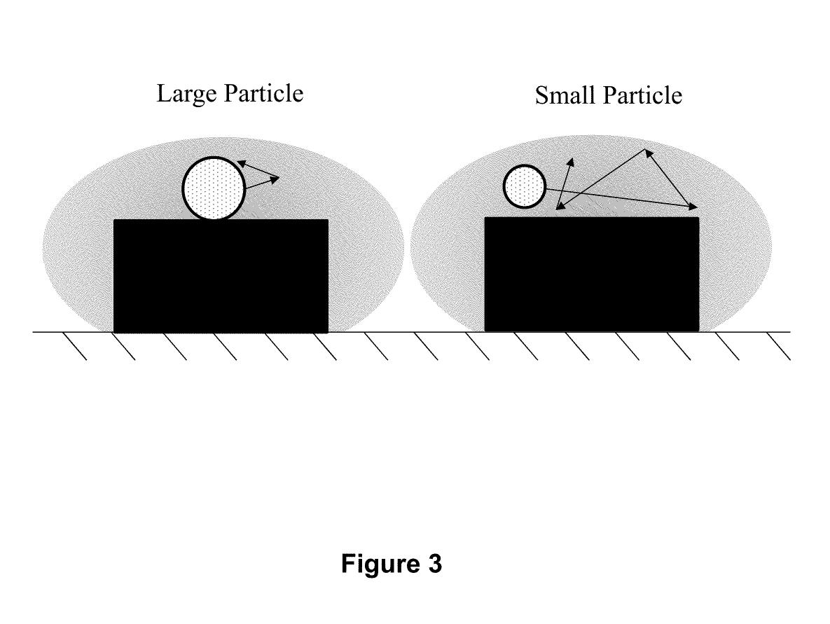

In one embodiment, a property of a substance is determined by measuring the motion of a substance. For example, in certain embodiments, the property is determined by measuring the motion of a trapped substance. The motion of the substance within the trap depends strongly on their size (size to the third power) and, to a lesser extent, their refractive index (refractive index to the first power). Thus, in some embodiments, the method comprises observing and tracking substance motion to accurately estimate substance properties. Larger substances exhibit less Brownian motion and are also more strongly held by the optical trap. Both effects serve to reduce substance motion within the trap. Conversely, smaller substances exhibit greater Brownian motion and are less strongly held by the optical trap (FIG. 3). The trap itself does not have discrete limits but instead can be thought of as a gradient of trapping force extending away from the optical structure. For a given optical power, larger particles will spend a greater percentage of their time near the center of the trap compared to smaller particles.

Substance motion can be observed at tracked by any method known in the art. For example, in one embodiment, substance motion is observed and tracked via tracking of a detectable label on the substance (i.e. a fluorescent tag). In another embodiment, substance motion is observed and tracked by observing the light coming out of the output of the waveguide. The interaction of the substance with a near field optical trap causes (1) light to be scattered, (2) a reduction in the amount of power coming out of the waveguide and/or (3) a shift in the resonance wavelength of the optical resonator (if one is being used). The closer the substance is to the trap, the greater the effect (i.e. the light scattering, power loss and resonance shift is increased).

The motion of the substance within the trap alternately brings the particle closer to and then further away from the resonator causing a respective increase and then decrease in the interaction effects. In one embodiment, the method comprises measuring the light at the output for a sufficient amount of time to observe substance motion within the trap and calculating at least one particle property, such as size, density or mass.

The physics of these measurements are dominated by trapping force, fluid viscous force, and Brownian energy. The trapping force, also known as the optical gradient force, is given by

$${F}_{grad} = \frac{2 π ∇ {I}_{o} α}{c} ,$$

where c is the speed of light, Iₒ is the incident light intensity, and α=3V(∈−∈ₘ)/(∈+2∈m), where V is the particle volume (proportional to the characteristic dimension size to the third power), and ∈₂ and ∈ₘ are the dielectric constants of the particle and material, respectively.

The fluid viscous force is given by:

F_{d}=6πμRν,

where μ is the viscosity, R is the characteristic dimension of the substance and v is the velocity of the substance.

The Brownian energy is given by k_{b}T, where k_{b} is the Boltzmann constant, and T is temperature of the fluid.

For stable traps, F_{grad}>F_{d} and F_{grad}>Brownian motion. Given that traps described herein are designed to operate this way, the trapping force is greatly enhanced for larger particles, due to the scaling of R³. Therefore, larger substances stay closer to the center of the trap over longer periods of time, while smaller substances spend more time farther from the center of the trap than a larger particle. Thus, in one embodiment, the method comprises observing the substance position, with respect to the center of the trap, in order to determine substance size. Substance size can be determined, for example, by referencing a database of relative positions for substances or particles of known size. Thus, in certain embodiments, observing the relative position of an unknown trapped substance is used in conjunction with a database to back calculate the size of the unknown substance. In another embodiment, observing and tracking the position of a trapped substance allows for determination of one or more dielectric properties of the trapped particle.

### Assays

The present invention also provides a method of using an optical trap to assay the relationship between substances. For example, in one embodiment, the method allows for evaluating if and/or how one or more substances may or may not bind to a trapped substance. In another embodiment, the method allows for evaluating if and/or how one or more substance may modulate the size, composition, or activity of a trapped substance. For example, the trapped substance may be a protein, nucleic acid, particle, protein coated particle, small molecule, and the like, while one or more other substances applied to the trapped substance may be a protein, nucleic acid, particle, protein coated particle, small molecule, compound, ion, and the like.

In one embodiment, the present invention provides a method for evaluating the binding of a trapped substance to one or more other substances. Trapping of a substance allows for the determination of binding affinity, binding kinetics, dissociation kinetics, binding stoichiometry, and the like. In some embodiments, the one or more other substances applied to the trapped substance comprise a test compound from a library.

In certain embodiments, the method comprises administering a substance of interest to an optical trap such that the trap immobilizes or traps the substance at or near a specified location, thereby providing a trapped substance. In order to evaluate the binding of the trapped substance to one or more other substances, the one or more other substances are then applied to the trapped substance. For example, in certain embodiments, the one or more other substances are provided in solution and are applied to the trapped substance using a fluidic delivery system. As described elsewhere herein, the flow of the solution comprising the one or more other substances may be specifically controlled or tuned to provide optimal assay conditions.

In certain embodiments, the assay comprises use of one or more substances that are labeled with a detectable label or detectable tag. In one embodiment, the substance of interest (i.e. the trapped substance) is labeled with a detectable label. In another embodiment, the one or more other substances (i.e. those substances subsequently applied to the trapped substance) are labeled with a detectable label. In another embodiment, both the trapped substance and the one or more other substances are labeled with a detectable label. Use of detectable labels allows for detection and/or quantification of binding of the trapped substance with the one or more other substances. For example, in certain embodiments, the method comprises using FRET techniques to measure binding and/or interaction of the substances. Thus, in certain embodiments, the method comprises use of known detection methods to observe an amount or detectable label or a change in the detectable label. As would be understood by those skilled in the art, any known detectable label may be used in the method. Exemplary labels include, but are not limited to, fluorescent labels, radioactive labels, ferromagnetic labels, paramagnetic labels, luminescent labels, electrochemiluminescent labels, phosphorescent labels, mass labels, Raman labels, molecular beacons, upconverting phosphors chromatic labels, and the like.

In one embodiment, the trapped substance and/or one or more other substances are labeled with a fluorescent label. Non-limiting examples of fluorescent labels include, green fluorescent protein (GFP), cyan fluorescent protein (CFP), yellow fluorescent protein (YFP), red fluorescent protein (RFP), orange fluorescent protein (OFP), eGFP, mCherry, hrGFP, hrGFPII, Alexa 488, Alexa 594, and the like. Fluorescent labels may also be photoconvertable such as for example kindling red fluorescent protein (KFP-red), PS-CFP2, Dendra2, CoralHue Kaede and CoralHue Kikume. In certain embodiments, the method comprises the use of a detector to determine the intensity, change in intensity, and/or shift in fluorescent wavelength emitted from the site of the trapped substance. Suitable detectors, for use in the present method, are well known in the art, and can include, but are not limited to fluorescence detectors, fluorescence microscopes, fluorescence spectrometers, and the like.

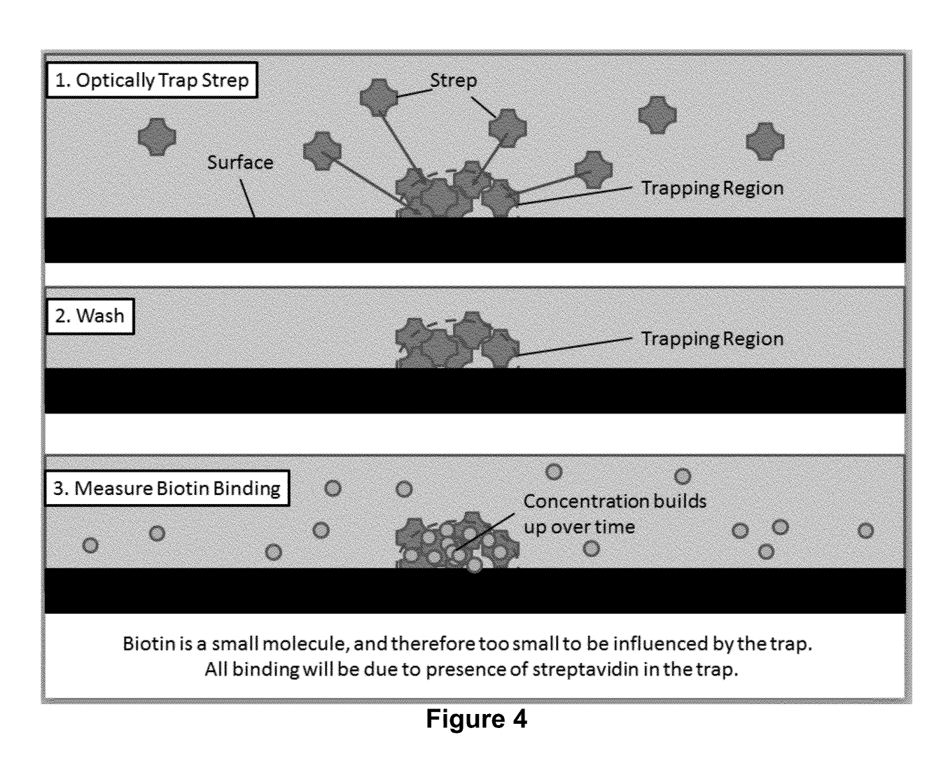

FIG. 4 illustrates a non-limiting example of optical trapping to bind one or more substances to one another. In FIG. 4, streptavidin is optically trapped to the surface at that trapping region. Any unbound streptavidin can be washed away while the optically trapped streptavidin remains. A binding partner (e.g. biotin) can then be allowed to come into contact with the streptavidin and the binding can be measured or monitored using traditional detection methods.

In another embodiment, the present invention provides a method for evaluating the modulation of a trapped substance by one or more other substances. For example, in one embodiment, the trapped substance is an aggregate, where the method comprises determining the extent at which one or more other substances induce the dissociation of the trapped aggregate. Dissociation of the aggregate may be determined, for example, through the loss of a detectable signal provided by a detectable label on the trapped aggregate. Alternatively, dissociation of the aggregate may be determined using one or more of the interrogation methods described elsewhere herein to determine the size of the aggregate over time.

In another embodiment, the method comprises determining the extent at which one or more other substances modulate the activity of a trapped substance. For example, in one embodiment, the trapped substance is an enzyme whose activity is determined by providing the enzyme with reactants and measuring the rate of product production. In certain embodiments, the method comprises providing a fluid to the trapped enzyme and determining the extent at which production of the enzyme product is altered. In certain embodiments, the fluid provided to the trapped enzyme comprises a protein, nucleic acid, small molecule, or the like. In another embodiment, the fluid provided to the trapped enzyme has an altered property (i.e. pH, osmolarity), that may or may not alter enzyme activity.

In another embodiment, the invention provides a method of detecting the presence of a substance in a sample. For example, the method comprises using an optical trap to immobilize an antibody or antibody coated particle, and providing a sample that may or may not comprise the substance of interest to the trapped antibody. This method provides an immunoassay method that, in certain instances, may be used to determine, for example, the presence of a particular biomarker.

### Systems

The present invention provides systems and devices for substance interrogation. In one embodiment, the system comprises an optical trap for trapping a substance of interest and one or more detectors. In certain embodiments, the optical trap comprises at least one structure from the group consisting of photonic wave guides, photonic crystal resonators, slot waveguides, and photonic tweezers. In certain embodiments, the detector is an instrument selected from the group consisting of a fluorescence microscope, a fluorescence detector, a fluorescence spectrometer, a light scattering detector, an optical sensor, a Raman microscope, a Raman spectrometer, and a spectrometer. In one embodiment, the system comprises a laser to provide optical energy to the optical trap. In one embodiment, the system comprises a fluidic delivery system to provide the substance of interest and/or other substances to the optical trap. In one embodiment, the system comprises a light source and focusing lens to provide interrogating light to a trapping region of the optical trap. In certain embodiments, the system further comprises a computer or other hardware with software suitable to program and/or control the system components. In one embodiment, the optical trap, fluidic delivery system, and at least one detector are all positioned on a single chip. In certain embodiments, a chip comprising the optical trap further comprises at least one on-chip sensor. Exemplary on-chip sensors include, but are not limited to, quartz crystal microbalances, cantilevers, electrochemical sensors, acoustic sensors, thermal sensors, impedance sensors, and whispering gallery mode optical sensors.

In one embodiment, the system comprises a plurality of optical traps, where each trap is tunable to perform a different characterization of a trapped substance. In one embodiment, each trap of the plurality of traps are tuned to trap a different size substance.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Multi-Chamber Particle Characterization

Production of pharmaceutical preparations often contains undesired contaminants within starting materials, intermediates, and/or final preparations. Presence of such contaminants can influence the overall effectiveness and safety of the product. While certain techniques may be able to provide information regarding particle contaminants greater than 2 μm in diameter, standard techniques are limited in interrogating the presence and/or properties of smaller contaminants.

As described herein, optical traps can be used in methods and systems to determine (1) how many particles there are in the solution, (2) what sizes these particles are and (3) what these particles composed of (e.g. rubber, protein, steel, oil)?

To investigate the presence and properties of small contaminants within a final pharmaceutical product, a fluid sample of the product is loaded into a chamber that contains a waveguide-based optical trap. This waveguide is exposed to the fluid so that the near field light emanating from the waveguide can interact with the fluid and particles within the solution. The system uses a three-step approach, where each step is accomplished in a different chamber of a microfluidic chip. In the first step, denoted as “Non-Trapping Analysis”, particles are analyzed as they flow near the waveguide and interact with the near field light but are not trapped. In the second step, denoted as “Small Ensemble Measurements”, numerous particles are actually captured by the waveguide and then analyzed. In the third step, denoted as “Single Particle Measurements”, numerous individual particles are captured, measured and released, freeing the trap for the next particle in a sequential manner.

Non-Trapping Analysis:

In Chamber 1, the power of the waveguide is low enough that it doesn't exert a strong enough force on nearby particles to trap them. For example, for spherical dielectric particles in liquid that are 0.2 to 2 μm in diameter with a refractive index of ˜1.5, a trap gradient strength of about 500V/μm² or lower is necessary so that particles do not get trapped. Instead, particles flowing through the chamber occasionally pass close enough to the waveguide to interact with the evanescent field and then continue on. The evanescent field decays exponentially and for the case of a silicon nitride waveguide in water carrying TE polarized 1064 nm light, 99% of the power is gone within 200 nm from the waveguide. During the brief time the particles pass through the evanescent field, they scatter some of the near-field light, thereby converting it into far-field light. Thus, the near-field light from the waveguide is used to interrogate the particles. Unlike the near-field light, this scattered far-field light travel long distances and are easily captured by photodetectors. An avalanche photodiode is used to collect the photons that are scattered by the contaminant particles as they pass by the waveguide. As an analogy, consider observing flies in a street light on a clear dark night. Without the flies, it is difficult to see the light from across the street because the light is aimed towards the ground and the clarity of the air prevents much light from scattering. Each fly that passes through the light looks like a tiny bright spot of light—the bigger the fly, the more light it scatters and the brighter the spot. In the present analysis, because the near field light has such a small interaction depth compared to far field light (e.g. a street light or even a laser shooting down into the fluid), much more precision is attained and solutions with higher contaminant concentrations are thus able to be examined without frequently interrogating two particles at once by coincidence. Each particle that passes into the evanescent field causes a short-lived spike of photons that reach the detector. Counting these spikes reveals the number of particles in solution.

Also, contaminant particles that are closer to the waveguide are bathed in more intense light and therefore scatter more light than those that are just slightly (e.g. 100 nm) farther away from the waveguide. Small particles like the ones considered here, move quickly with Brownian motion. Their movements take them farther from or closer to the waveguide—scattering respectively less and then more light. Thus, as particle size decreases, particle movement becomes quicker and more erratic. Thus, particle size is estimated based on the fingerprint of their scattering, which indicates their Brownian motion, which, in turn, indicates their size.

Small Ensemble Measurements:

After the particles have undergone Non-Trapping Analysis, they progress through the microfluidic system into Chamber 2, which is equipped to do Small Ensemble Measurements. In this chamber, there is an exposed waveguide similar to that in Chamber 1. In this case, however, the waveguide is equipped with an evanescently coupled 1D photonic crystal resonator. The wavelength of the coupled laser is tuned so that the resonator is active and amplifying the optical power in order to more effectively trap particles. Only particles within a specific size range are trapped. Larger particles are swept away by drag forces caused by fluid flow. Particles that are too small will not be sufficiently influenced by the trap and also flow by. Studies have shown that 0.2 μm polystyrene nanoparticles are easily trapped with waveguides or photonic resonators. Internal testing has revealed that approximately 0.5 to 20 mW are required to trap substances in this range. The flow rate and laser strength is tuned so that only contaminants that are between 0.2 μm to 2 μm are trapped.

When the particles enter the chamber, a portion of the suspended contaminants are captured by the evanescent field emanating from the photonic crystal. Over time, the number of captured contaminant particles continues to build up and cover the photonic crystal. After a fixed period of time, a suite of three measurements on the trapped particles begins. (1) The first measurement is to observe the light coming out of the waveguide. (2) The second measurement is to carry out Raman spectroscopy on trapped particles using the trapping wavelength to induce the Raman shift. (3) The third measurement is to carry out Raman spectroscopy on the trapped particles using an outside laser aimed at the trapping site to induce a Raman shift.

Measurement Technique 1:

Captured particles have the effect of perturbing the resonance wavelength of the photonic crystals (generally red-shifting them). This affects the power coming out of the waveguide, which is measurable. If the incoming light was exactly tuned to the resonance wavelength, a red shift caused by particles accumulating in the trap causes the power measured at the output to decrease. The more particles (or the larger the particles trapped), the more the power shift. This technique therefore gives an estimation of the number and size of the particles being trapped.

Measurement Technique 2:

The trapped particles also absorb and scatter some of the near field light emanating from the waveguide. This is used to get a Raman spectrometry signal from the trapped particles. This signal is captured by a spectrometer and is then used to determine the particle type (i.e. whether it's a piece of metal or a protein). Raman spectrometry can determine whether a particle is an aggregate of the therapeutic or if it is some other substance.

Measurement Technique 3:

The trapped particles are also interrogated by an external light source to generate a Raman signal similar to that described above in technique 2. The advantage of this technique is that it allows for the use of different wavelengths to interrogate the trapped particles.

Single Particle Measurements:

After the particles have undergone Small Ensemble Measurements, they progress through the microfluidic system into the third and final chamber, which is equipped to do Single Particle Measurements. In this chamber, there is an exposed waveguide with a resonator similar to the one in the previous chamber. In this case, the resonator is in-line with the waveguide, which produces the strongest trap and is capable of trapping the smallest particles. In this chamber the three measurement techniques described above in the previous chamber are used here. However in this chamber smaller particles (i.e. <0.2 μm) are measured individually. This is the most time consuming analysis, but it offers true single-particle analysis.

### Example 2

### Binding kinetics

The binding kinetics of a trapped substance is measured. Using a near-field optical trap within a channel, the binding rate of a fluorescently labeled small molecule (B) to a protein (A) is measured. The channel and materials are passivated by flowing in a blocking buffer (such as Bovine Serum Albumin). The trap is then activated. A protein is applied (i.e. by flowing the protein into the channel) and the protein is captured by the trap. The unbound protein is washed away with an appropriate buffer. Molecule B is flowed into the trap at a controlled rate. While molecule B is flowing in, the fluorescence of the trap is measured, thereby by providing the binding rate of B to protein A. Unbound B can also be washed away. Another fluorescent measurement of the trap is made to determine the affinity of molecule B to protein A. The protein bound to or not bound to molecule B is optionally eluted and analyzed.

### Example 3

### Dissociation of Aggregates/Complexes

Using a near-field optical trap within a channel, testing the concentration of chemical B to disassociate fluorescently labeled protein aggregate A is measured/observed. The channel and materials are passivated by flowing in a blocking buffer (such as Bovine Serum Albumin). The trap is then activated. The protein aggregate is applied (i.e. by flowing the aggregate into the channel), which is captured by the trap. The unbound proteins aggregates are washed away with an appropriate buffer. A solution containing molecule B is applied at a controlled rate with steadily increasing concentration. While molecule B is being fluidically delivered to the trap, the fluorescence of the trap is measured. After a period of time of flowing in molecule B, a high enough concentration is present to dissociate the protein aggregate, causing the individual components/subunits to break apart and flow away. Molecule B is then identified as a molecule that can dissociate an aggregate or complex. The concentration required to dissociate an aggregate or complex is also determined. The remaining complex is optionally eluted, collected and analyzed.

### Example 4

### Protein Activity as a Function of pH

Using a near-field optical trap within a channel, testing the effect of pH on the activity of protein A is measured. The channel and materials are passivated by flowing in a blocking buffer (such as Bovine Serum Albumin). The trap is then activated. Protein A is applied (i.e. by flowing protein A into the channel), which is captured by the optical trap. The unbound protein is washed away with an appropriate buffer. Protein A's reactants are then applied. The products are measured by spectrometry (UV/Vis). The buffer containing the reactants is slowly altered so that a range of pH is used. For example, starting with a pH of 6 and slowly increasing the pH over time to a pH of 9. While the pH is changing, the rate of reactant production of the enzyme is measured. The effect of pH on the protein activity is determined and/or recorded.

### Example 5

### Immunoassay

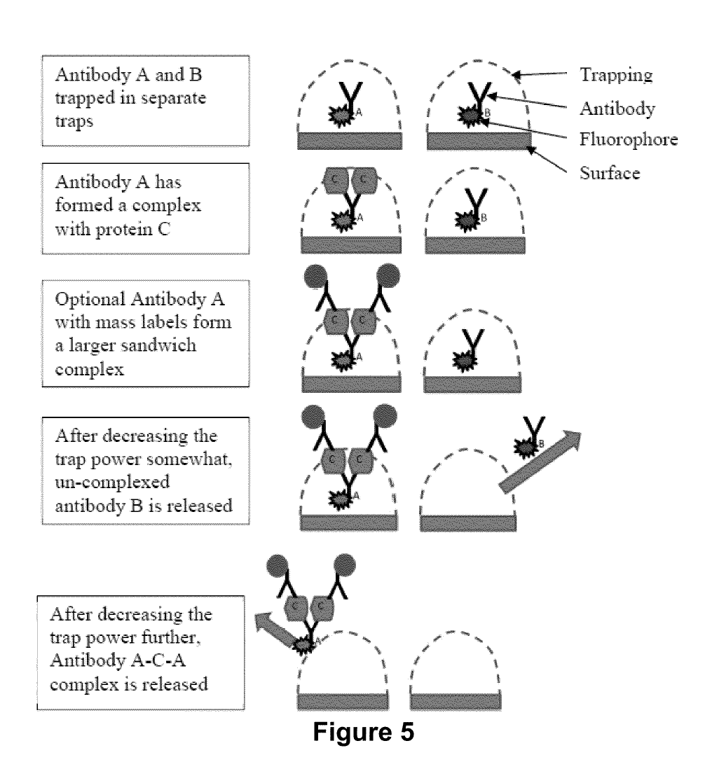

Using a near-field optical trap within a channel as an immunoassay to measure the presence of protein C which binds to red fluorescence-labeled antibody A, but not green fluorescence-labeled antibody B. The channel and materials are passivated by flowing in a blocking buffer (such as Bovine Serum Albumin). Two traps (Trap 1 and Trap 2) are then activated. Antibody A and antibody B are applied (i.e. by flowing the antibodies in separate reagent streams) such that antibody A gets trapped in trap 1 and antibody B gets trapped in trap 2. Antibody A and B can be kept separate by using two different channels or by using multiple laminar streams. The unbound antibodies are washed with an appropriate buffer. An “unknown” sample is applied to test it for the presence of protein C for a sufficient time for C, if present, to bind to the Antibody A or B. The optical trap is washed to remove any unbound sample. A sandwich assay an also be used. While monitoring the fluorescence of trap 1 and trap 2, the power of the trap is slowly decreased. As the trap power decreases, the smaller particles first elute off, and then the larger ones elute off. Any complexes with antibody A that have formed as a result of the presence of protein C will be larger than un-complexed antibody B. Thus the individual antibody B will elute off at a higher power than the complex of antibody A+protein C. If a sandwich assay is used, the mass of the protein C complex will increase, increasing the measurement metric. That is without the sandwich assay, the complex will be [A]-[C]. With the sandwich assay, the complex will be [A]-[C]-[A+mass]. This optional complex will be much larger and will elute off at an even lower power than [A]-[C] alone. This makes the measurement more sensitive. FIG. 5 illustrates a non-limiting example of this method.

### Example 6

### Systems for Substance Measurement

An example of a system capable of performing measurements of one or more substances of interest is described as follows. The system provides for trapping of a group of substances in a fluid using a waveguide. The near field light from the waveguide traps the substance and serves as the excitation source for Raman spectroscopy. The group of substances is then released.

To carry out these measurements, the system comprises a power supply, fiber-coupled semiconductor laser, an optical isolator to protect the laser from back scatter, photodiode signal digitizer, syringe pump, computer interface hardware, AC/DC power supply for USB ports for computer interface, a single mode polarization maintaining fiber optic that connects to a silicon nitride waveguide on a silicon chip. The chip sits in a plastic carrier which assists handling and fits into a holder that connects the fluid lines from the syringe pump to the chip. The chip has a laser cut adhesive gasket that defines a microfluidic channel which is completed with an optical cover slip. The chip and mount are placed under an objective lens that collects the Raman signal from the trapped particles. The light goes through a high pass filter that blocks the scattered excitation light and then enters a spectrometer for the measurement.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of measuring at least one property of a substance comprising:
positioning a substance in the vicinity of near-field light of an optical trap;
directing light from a light source to the substance;
detecting the effect of the light on the substance; and
measuring at least one property of the substance based on the detected effect.

2. The method of claim 1, wherein the effect of the light is selected from the group consisting of light scattered by the substance, light emitted by the substance, and light absorbed by the substance.

3. The method of claim 1, further comprising:
immobilizing the substance at a location using the optical trap, thereby forming a trapped substance.

4. The method of claim 1, wherein the optical trap comprises at least one structure selected from the group consisting of optical fibers, photonic waveguides, slot waveguides, plasmonic tweezers, photonic crystal resonators, ring resonators, toroidal resonators, Whispering Gallery Mode Resonators, and Fabry Perot resonators.

5. The method of claim 1, wherein the substance is a substance selected from the group consisting of a molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome.

6. The method of claim 1, wherein the at least one property of the substance is a property selected from the group consisting of size, structure, chemical composition, refractive index, electrical impedance, electrical permittivity, mass, density, temperature, diffusion coefficient, shape, protein folding state, solubility, crystallinity, enzymatic activity, binding activity, binding kinetics, and dissociation kinetics.

7. The method of claim 1, wherein the light source is the near-field light of the optical trap.

8. The method of claim 1, wherein the light source is an external light source.

9. The method of claim 2, wherein detecting the scattered light comprises detecting the amount of the scattered light.

10. The method of claim 2, wherein detecting the scattered light comprises detecting the amount and wavelength of the scattered light.

11. The method of claim 2, wherein detecting the amount of scattered light comprises the use of a detector selected from the group consisting of a light scattering detector, spectrometer, Raman spectrometer, photodiode, charged coupled device (CCD), spectrum analyzer, interferometer, ellipsometer, integrating sphere, and photomultiplier.

12. The method of claim 1, wherein measuring the property of the substance comprises measuring the motion of the substance.

13. The method of claim 3, further comprising releasing the trapped substance.

14. A method of measuring the binding activity of a substance comprising;
immobilizing the substance at a location using an optical trap, thereby forming a trapped substance;
contacting the trapped substance with one or more test substances; and
detecting the binding of the strapped substance with one or more test substances.

15. The method of claim 14, wherein the optical trap comprises at least one structure selected from the group consisting of optical fibers, photonic waveguides, slot waveguides, plasmonic tweezers, photonic crystal resonators, ring resonators, toroidal resonators, Whispering Gallery Mode Resonators, and Fabry Perot resonators.

16. The method of claim 14, wherein the trapped substance is a substance selected from the group consisting of a molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome.

17. The method of claim 14, where in the test substance is a substance selected from the group consisting of a molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome.

18. The method of claim 14, wherein the method measures the binding kinetics between the trapped substance and the test substance.

19. The method of claim 14, wherein the method measures the binding affinity between the trapped substance and the test substance.

20. The method of claim 14, wherein at least one of the trapped substance and test substance are labeled with a detectable label, and wherein the detecting of binding comprises detecting a detectable signal from the detectable label.

21. The method of claim 14, wherein the detectable label is selected from the group consisting of fluorescent labels, radioactive labels, ferromagnetic labels, paramagnetic labels, luminescent labels, electrochemiluminescent labels, phosphorescent labels, mass labels, Raman labels, molecular beacons, upconverting phosphors and chromatic labels.

22. The method of claim 14, wherein the test substance is contacted with the trapped substance by flowing the test substance to the trapped substance.

23. A method of identifying a modulator of a substance comprising:
immobilizing the substance at a location using an optical trap, thereby forming a trapped substance;
contacting the trapped substance with one or more test substances; and
measuring a property of the trapped substance, wherein a change in the property of the trapped substance when contacted with the test substance indicates that the test substance is a modulator of the substance.

24. The method of claim 23, wherein the optical trap comprises at least one structure selected from the group consisting of optical fibers, photonic waveguides, slot waveguides, plasmonic tweezers, photonic crystal resonators, ring resonators, toroidal resonators, Whispering Gallery Mode Resonators, and Fabry Perot resonators.

25. The method of claim 23, wherein the trapped substance is a substance selected from the group consisting of molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome.

26. The method of claim 23, wherein the test substance is a substance selected from the group consisting of molecule, compound, nucleic acid, peptide, protein, antibody, enzyme, quantum dot, nanotube, particle, virus, bacteria, cell, protein complex, carbohydrate, lipoparticle, vesicle, microparticle, oil droplet, and liposome.

27. The method of claim 23, wherein the property of the trapped substance is a property selected from the group consisting of size, structure, chemical composition, enzymatic activity, binding activity, binding kinetics, and dissociation kinetics.

28. The methods of claim 23, wherein the test substance is contacted with the trapped substance by flowing the test substance to the trapped substance.

29. A system for measuring a property of a substance comprising:
at least one optical trap; and
at least one detector for measuring the property of the substance.

30. The system of claim 29, further comprising a microfluidic delivery system.

31. The system of claim 29, wherein the at least one optical trap comprises at least one structure selected from the group consisting of optical fibers, photonic waveguides, slot waveguides, plasmonic tweezers, photonic crystal resonators, ring resonators, and toroidal resonators.

32. The system of claim 29, wherein the at least one detector comprises a detector selected from the group consisting of fluorescence microscopes, fluorescence detectors, fluorescence spectrometers, light scattering detectors, optical sensors, Raman microscopes, Raman spectrometers, spectrometers, photodiodes, charged coupled devices (CCDs), Complementary metal-oxide-semiconductor (CMOS) cameras, spectrum analyzers, interferometers, ellipsometers, integrating spheres, and photomultipliers.

33. The system of claim 29, further comprising an external light source.

34. The system of claim 29, wherein the at least one optical trap comprises at least one power source.

35. The system of claim 34, wherein the power source is an optical power source configured to provide optical power to the optical trap.

36. The system of claim 29, further comprising at least one sensor selected from the group consisting of quartz crystal microbalances, cantilevers, electrochemical sensors, acoustic sensors, thermal sensors, impedance sensors, and whispering gallery mode optical sensors.

37. The system of claim 29, wherein the at least one optical trap is patterned on substrate selected from the group consisting of a silicon substrate, glass substrate and polymer substrate.
